(19) United States
(12) Patent Application Publication
(10) Pub. No.: US 2023/0250400 A1
(43) Pub. Date: Aug. 10, 2023

(54) METHODS FOR ENGINEERING HUMAN PLURIPOTENT STEM CELLS FOR INSULIN PRODUCTION

(71) Applicant: Xingxin Wang, Toronto (CA)

(72) Inventor: Xingxin Wang, Toronto (CA)

(21) Appl. No.: 18/182,137

(22) Filed: Mar. 10, 2023

Related U.S. Application Data
(63) Continuation-in-part of application No. 16/951,220, filed on Nov. 18, 2020, now abandoned.

(51) Int. Cl. C12N 5/074; C07K 14/72; C07K 14/62

(52) U.S. Cl. CPC C12N 5/0696; C07K 14/721; C07K 14/62; C12N 2506/1384; C12N 2510/02; C12N 2500/32; C12N 2500/44; C12N 2501/115; C12N 2501/2303; C12N 2501/2317; C12N 2533/54

(57) ABSTRACT

The present disclosure provides an in vitro method for preparation of human pluripotent stem cells (HPSCs) from human adipocyte-derived stem cells (ADSCs) without any genetic engineering techniques and without involving any exogenous gene elements, plasmid or transcription factors and the so obtained HPSCs are referred to as directly-generated human pluripotent stem cells (dgHPSCs). The present invention further provides an in vitro method for insulin production from the dgHPSCs by means of single- or co-transduction with human estrogen-related receptor gamma (ERRγ) gene by the lentivirus vector pWPI/ERRγ encoding the human ERRγ gene and/or with human insulin (INS) gene by a lentivirus vector, pWPI/INS encoding the human INS gene, where the insulin secreted by such co-transduced cells is higher than singly transduced cells. The present invention also provides an in vitro method for insulin production in a glucose-concentration responsive manner involving single transduction of the dgHPSCs with the human ERRγ gene.

### CROSS REFERENCE TO RELATED APPLICATIONS

The current application is a continuation-in-part of the U.S. patent application Ser. No. 16/951,220 filed Nov. 18, 2020.

### FIELD OF THE INVENTION

The present disclosure relates generally to human pluripotent stem cells. More specifically, the present disclosure describes methods for engineering human pluripotent stem cells for insulin production.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Since the discovery of insulin, daily insulin injection has been generally used for treatment of diabetes with absolute insulin deficiency. However, with the progression of symptoms, exogenous insulin administration may need to be intensified with more dosages. Moreover, it gets even worse when only administration of insulin cannot maintain blood glucose levels within the narrow physiological range that protects diabetes patients from development of various diabetic complications due to insulin injection which cannot exactly mimic pancreatic β cells to adjust insulin secretion in response to varying blood glucose levels. Thus, currently available therapies for diabetes have limited effects in preventing the progression of diabetes complications and repairing existing tissue damages. Therefore, improvements of the current diabetes therapies with novel strategies are highly anticipated and needed. Restoring the normal functions of pancreatic β cells is critical and a way to finally cure diabetes and protect patients from diabetic complication development.

To generate insulin-producing pancreatic β cells in vitro, human embryonic stem cells (hESCs) and human induced pluripotent stem cells (hiPSCs) provide unlimited cell sources for transplantation therapy for diabetes. So far, a number of groups have generated immature or mature human insulin-producing pancreatic β cells from hESCs and hiPSCs. Although the protocols to generate pancreatic β cell-like cells are complicated and challenging, these achievements are encouraging and promising with the potential for better treatment for human diabetes, and further effort in development of the insulin-producing stem cells toward clinical diabetes therapy is highly anticipated.

Estrogen-related receptor γ (ERRγ) is a master regulator of β cell maturation in vivo. Forced expression of ERRγ in hiPSC-derived β-like cells enables glucose-responsive secretion of human insulin in vitro and can further restore the glucose homeostasis in type 1 diabetes mouse models after transplantation, without the need for kidney capsule maturation, and to achieve functionality immediately. A US patent application, US20150368667 A1 provides a method of inducing β cell maturation from embryonic or induced pluripotent stem cell-derived β-like cells, the method involved introducing ERRγ expression by engineering the cells via ERRγ gene vectors, the vector could be lentiviral vector or adeno associated viral vector. Due to ethical concerns about using embryonic stem cells and therapeutic safety concerns about genetic engineering techniques, which may cause host genome aberration, used for generating induced pluripotent stem cells, it becomes a novel approach to generate induced pluripotent stem cells by non-genetic engineering techniques, and it may potentially be applied into stem cell therapy for diabetes.

Human adipose-derived stem cells (hADSCs) were confirmed to have the potential to differentiate toward osteogenic, adipogenic, myogenic, chondrogenic, and putative neurogenic cells. Sun et al., reported the successful induction of hiPSCs from hADSCs with lentivirus containing human Oct4, Sox2, Klf4, and c-MYC genes. However, the use of oncogene c-MYC gene as one of the inducing factors remains to be a potential concern for clinical application of these hADSC-derived iPSCs. The article Qu et al., provides that adipose-derived stem cells (ADSCs) can be reprogrammed into induced pluripotent stem cells by a plasmid vector which does not integrate into the host genome. However, introducing plasmid to a host cell poses a risk of integration of exogenous DNA elements into the host genome as has been reported by Wang et al., using a quantitative gel-purification assay for integration, the study identified four independent integration events after plasmid intramuscular injection and electroporation, and sequencing of the insertion sites suggested a random integration process. Although the integration of plasmid DNA into host cells has not been widely reported, from the view of human gene therapy, it reasonably says that the safety concern cannot be ruled out. In Qu et al., four transcription factors Oct4, Sox2, Klf4 and C-Myc were fused into the plasmid and expressed in the host cells, all of the four genes were reported to be either oncogenes, such as c-Myc and Klf4, or genes that exhibit high expression in various types of cancer, such as Oct3/4 and Sox2.

The present invention addresses the above-identified need in the art for insulin production, where the production is glucose concentration-responsive while addressing the safety concerns pointed out in the discussed prior art and also with an effort to reduce the chance of causing aberrational epigenetic events in the reprogrammed stem cells by using an induction medium.

### SUMMARY OF THE INVENTION

The present invention provides a unique and novel approach with the use of a specific induction medium for preparation of human pluripotent stem cells (HPSCs) from non-pluripotent stem cells, i.e., human adipocyte-derived stem cells (ADSCs) without involving any exogenous gene elements, neither plasmid nor transcription factors referred to as directly-generated human pluripotent stem cells (dgHPSCs). The present invention further provides an in vitro method for insulin production from dgHPSCs by means of co-transduction with human estrogen-related receptor gamma (ERRγ) gene by the lentivirus vector pWPI/ERRγ encoding the human ERRγ gene and with human insulin (INS) gene by a lentivirus vector, pWPI/INS encoding the human INS gene, where the insulin secreted by such co-transduced cells is higher in comparison to dgHPSCs singly transduced with the human ERRγ gene. Additionally, the present invention provides an in vitro method for insulin production from the aforementioned dgHPSCs in a glucose-concentration responsive manner involving transduction with the human ERRγ gene.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWING

The accompanying drawings are included to provide a further understanding of the invention, and are incorporated in and constitute a part of the present invention and, together with the description, serve to explain the principle of the invention.

In the drawings,

FIG. 1 illustrates a schematic flow chart representation of the overall process in terms of steps for the claimed method for engineering human pluripotent stem cells (HPSCs) for insulin production and secretion of potentially therapeutic levels of insulin protein by transduction of ERRγ gene, insulin gene, and co-transduction of ERRγ gene along with insulin gene, as well as in a glucose concentration-responsive manner by transduction of ERRγ gene, according to embodiments of the present disclosure.

FIG. 2 depicts an inverted phase contrast microscope image of cell morphology of isolated mononuclear cells from lipoaspirate, according to embodiments of the present disclosure.

FIG. 3A and FIG. 3B depict phenotypic characterization of hADSCs by flow cytometry. In consistence with the most commonly reported hADSCs cell surface markers, the isolated cells demonstrated phenotype of hADSCs which is illustrated in FIG. 3A for CD14 (panel (a)), CD19 (panel (b)), CD34 (panel (c)), CD45 (panel (d)), and HLA-DR (panel (e)) as negative staining; and in FIG. 3B for CD44 (panel (f)), CD73 (panel (g)), CD90 (panel (h)), CD105 (panel (i)), and CD166 (panel (j)) as positive staining. “M1” in the flow cytometry histogram here is a marker placed to designate the range for positive peaks.

FIG. 4 depicts microscopic images of human adipose-derived stem cells (hADSCs) with Oil red O staining in panel (a) showing these cells are rich in fat droplets suggesting that the cells possessed adipocyte differentiation potential in vitro, and intense Alizarin red staining in panel (b) after 3 weeks showed mineralized nodules suggesting that these hADSCs of the present disclosure possessed osteoblasts differentiation potential in vitro, according to embodiments of the present disclosure.

FIG. 5 depicts a microscopic image of human pluripotent stem cells (HPSCs) marker, TRA-1-60 immuno-fluorescent stain to show the TRA-1-60 positive cells as the in vitro produced human pluripotent stem cells (HPSCs) which are produced in the present disclosure by treating human adipose-derived stem cells (hADSCs) with an induction medium without applying any genetic engineering techniques for at least five passages referred to as P5 to obtain P5 hADSCs and testing the cultured cells with the TRA-1-60 immuno-fluorescent stain, where said TRA-1-60 positive cells are HPSCs derived from P5 hADSCs referred to as directly-generated human pluripotent stem cells (dgHPSCs), according to embodiments of the present disclosure.

FIG. 6 depicts a microscopic image of insulin fluorescent immunochemistry staining of the directly generated human pluripotent stem cells, where insulin immunofluorescent stain in lentivirus vector pWPI/EERγ transduced dgHPSC cells is presented, according to embodiments of the present disclosure. Panel (a) presents the DAPI nuclear stain in the nucleus of the non-transduced dgHPSC cells but there is no immunofluorescent TRITC stain revealed in the cytoplasm of those non-transduced dgHPSC cells indicating no insulin production in the cells; while panel (b) presents the DAPI nuclear stain in the nucleus of pWPI/EERγ transduced dgHPSC cells and immunofluorescent TRITC stain in the cytoplasm of these transduced dgHPSC cells indicates insulin production in the transduced cells, according to embodiments of the present disclosure.

Unless otherwise specifically noted, articles depicted in the drawings are not necessarily drawn to scale.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

The present disclosure includes many aspects and features. Moreover, while many aspects and features relate to, and are described in the context of method for engineering human pluripotent stem cells for insulin production having future potential diabetes therapy by co-transduction, embodiments of the present disclosure are not limited to use only in this context.

Since the discovery of insulin, daily insulin injection has been generally used for treatment of diabetes with absolute insulin deficiency. However, with the progressing symptoms, exogenous insulin administration may need to be intensified with more dosages; and even worse, only administration of insulin cannot maintain blood glucose levels within the narrow physiological range that protects patients from development of various diabetic complications which is due to the fact that insulin injection alone cannot exactly mimic pancreatic β cells to adjust insulin secretion in response to varying blood glucose levels. Currently, the available therapies for diabetes have limited effects in preventing the progression of diabetes complications and repairing existing tissue damages. Therefore, improvements of the current diabetes therapies as well as novel methods for insulin production for potential diabetes therapy with novel strategies are highly anticipated, to restore the normal functions of pancreatic β cells which is critical to finally cure diabetes and protect patients from diabetic complication development.

The present disclosure provides a novel approach, which can directly generate human pluripotent stem cells, from non-pluripotent stem cells, i.e., human adipose-derived stem cells (hADSCs) with culturing and inducing them in specific induction medium without applying any genetic engineering techniques to generate pluripotent stem cells referred to as directly-generated human pluripotent stem cells (dgHPSCs). The aforesaid dgHPSCs are then genetically engineered as disclosed in the present disclosure to achieve potentially therapeutic levels of insulin production in particular by co-transduction with human estrogen-related receptor gamma (ERRγ) and insulin (INS) genes via lentivirus vector transductions to simultaneously introduce the human ERRγ gene and the human INS gene in the dgHPSCs to obtain co-transduced dgHPSCs referred to as dgHPSCs+ERRγ+INS cells. Further, the dgHPSCs transduced singly with the human ERRγ gene via lentivirus vector transduction, referred to as dgHPSCs+ERRγ cells, provide glucose-concentration responsive insulin secretion.

The instant disclosure provides an in vitro method for insulin production seeking to potentially provide a novel concept of developing an insulin therapy for diabetes, and it centers on a method for developing a human pluripotent stem cell line, dgHPSC as aforementioned, that is produced from hADSCs induced while in culture by a non-genetic modification approach with the use of a specific induction medium, and the so-obtained dgHPSCs are transduced by a lentivirus vector carrying human ERRγ gene as well as co-transduced by lentivirus vectors carrying human ERRγ and INS genes, respectively. Expression of the human ERRγ gene or co-expression of the human ERRγ and INS genes appears to synergistically promote the synthesis and secretion of human insulin at the stem cell state, and thus the present disclosure provides cells that do not need to differentiate into matured β-like cells to produce and secrete insulin protein with potential for diabetes therapy. As a future application of the present disclosure, transplantation of the transduced or co-transduced dgHPSC cells as disclosed herein into diabetic patients may present a promising cell therapy for diabetes.

An embodiment of the present disclosure provides an in vitro method for producing human pluripotent stem cells (HPSCs) for insulin production, the method comprising the steps of: (i) obtaining a lipoaspirate from a human; (ii) preparing human adipose-derived stem cells (hADSCs) from the lipoaspirate of step (i) involving isolation and single cell clone selection of adipose stem cells to obtain a selected clone followed by proliferation and expansion of cells of the selected clone to obtain the hADSCs; (iii) inducing and growing the hADSCs from step (ii) in an induction medium for at least five passages referred to as P5 to obtain P5 hADSCs; (iv) culturing the P5 hADSCs of step (iii) in the induction medium without applying any genetic engineering techniques to obtain cultured cells and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs of step (iii) referred to as directly-generated human pluripotent stem cells (dgHPSCs); (v) genetically engineering the dgHPSCs of step (iv) in a manner selected from a group consisting of: by transduction of said dgHPSCs with the human estrogen-related receptor gamma (ERRγ) gene by a lentivirus vector, pWPI/EERγ encoding the human ERRγ gene to obtain transduced dgHPSCs referred to as dgHPSCs+ERRγ cells, which are first of the groups of HPSCs for insulin production, and by co-transduction of said dgHPSCs with said ERRγ gene by the lentivirus vector pWPI/ERRγ and with the said INS gene by a lentivirus vector, pWPI/INS encoding the human INS gene to simultaneously introduce the human ERRγ gene and the human INS gene in the dgHPSCs of step (iv) to obtain co-transduced dgHPSCs referred to as dgHPSCs+ERRγ+INS cells, which are second of the groups of HPSCs for insulin production, wherein the co-transduced dgHPSCs referred to as dgHPSCs+ERRγ+INS cells show co-expression of the human ERRγ gene and the human INS gene to synergistically promote the synthesis and secretion of human insulin at the stem cell state, and wherein the co-transduced dgHPSCs referred to as dgHPSCs+ERRγ+INS cells transduced with both the human ERRγ gene and the human INS gene secrete a higher level of human insulin compared to dgHPSCs transduced with only the human ERRγ gene referred to as dgHPSCs+ERRγ cells.

An alternate embodiment of the in vitro method for producing HPSCs for insulin production as disclosed herein, wherein the lipoaspirate from a human of step (i) as disclosed hereinabove is obtained by collecting a lipoaspirate by an abdominal adipose tissue liposuction procedure performed in a volunteer human donor.

An alternate embodiment of the in vitro method for producing HPSCs for insulin production as disclosed herein, wherein the human adipose-derived stem cells (hADSCs) are prepared in step (ii) as disclosed hereinabove from the lipoaspirate of step (i) as disclosed hereinabove by a method comprising the steps of: (a) washing the lipoaspirate of step (i) as disclosed hereinabove with Dulbecco's phosphate buffered saline (DPBS) and centrifuging them to remove red blood cells into the suspension and for obtaining a cell pellet; (b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube; (c) repeating the digestion with collagenase as in step (b) until the lipoaspirate of step (i) as disclosed hereinabove is completely digested and to isolate mononuclear cell layer in each such repetition to obtain a composite of mononuclear cell layer; and (d) culturing the composite of mononuclear cell layer obtained in step (c) in StemPro MSC SFM XenoFree medium with 1% volume by volume (V/V) of non-essential amino acid, 10 ng/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

An alternate embodiment of the in vitro method for producing HPSCs for insulin production as disclosed herein, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acids, 1:1000 volume by volume (V/V) ratio of 2-Mercaptoethanol to induction medium, 10 ng/ml basal fibroblast growth factor (bFGF), 3 ng/ml weight by volume (W/V) of interleukin-3, and 5 ng/ml weight by volume (W/V) of interleukin-17.

An alternate embodiment of the in vitro method for producing HPSCs for insulin production as disclosed herein, wherein the induction medium in step (iii) as disclosed hereinabove is changed every other day till the hADSCs reach confluence before passages up to the at least five passages referred to as P5 to obtain the P5 hADSCs, and wherein the hADSCs of step (iii) as disclosed hereinabove until obtaining of said P5 hADSCs are grown in 10-cm cell culture dishes coated with 0.1% weight by volume (W/V) of gelatin.

An alternate embodiment of the in vitro method for producing HPSCs for insulin production as disclosed herein, wherein the dgHPSCs of step (iv) as disclosed hereinabove can be viably passaged for at least twenty passages.

An embodiment of the present disclosure provides an in vitro produced human pluripotent stem cells (HPSCs) for insulin production comprising: HPSCs produced from human adipose-derived stem cells (hADSCs) by treating said hADSCs with an induction medium without applying any genetic engineering techniques for at least five passages referred to as P5 to obtain P5 hADSCs and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs referred to as directly-generated human pluripotent stem cells (dgHPSCs); and co-transduction of said dgHPSCs with the human estrogen-related receptor gamma (ERRγ) gene by the lentivirus vector pWPI/ERRγ encoding the human ERRγ gene and with human insulin (INS) gene by a lentivirus vector, pWPI/INS encoding the human INS gene to simultaneously introduce the human ERRγ gene and the human INS gene in the dgHPSCs to obtain co-transduced dgHPSCs referred to as dgHPSCs+ERRγ+INS cells, wherein the hADSCs are produced from a lipoaspirate from a human involving isolation and single cell clone selection of adipose stem cells to obtain a selected clone followed by proliferation and expansion of cells of the selected clone to obtain the hADSCs, wherein the dgHPSCs are alternatively genetically engineered by transduction of said dgHPSCs with the human estrogen-related receptor gamma (ERRγ) gene only by a lentivirus vector, pWPI/EERγ encoding the human ERRγ gene to obtain transduced dgHPSCs referred to as dgHPSCs+ERRγ cells, and wherein the co-transduced dgHPSCs referred to as dgHPSCs+ERRγ+INS cells transduced with both the human ERRγ gene and the human INS gene secrete a higher level of human insulin compared to dgHPSCs transduced with only the human ERRγ gene referred to as dgHPSCs+ERRγ cells.

An alternate embodiment of the in vitro produced human pluripotent stem cells (HPSCs) for insulin production as disclosed herein, wherein the lipoaspirate from a human is obtained by collecting a lipoaspirate by an abdominal adipose tissue liposuction procedure performed in a volunteer human donor.

An alternate embodiment of the in vitro produced human pluripotent stem cells (HPSCs) for insulin production as disclosed herein, wherein the human adipose-derived stem cells (hADSCs) are prepared from the lipoaspirate by a method comprising the steps of: (a) washing the lipoaspirate with Dulbecco's phosphate buffered saline (DPBS) and centrifuging them to remove red blood cells into the suspension and for obtaining a cell pellet; (b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube; (c) repeating the digestion with collagenase as in step (b) until the whole of the lipoaspirate is completely digested and to isolate mononuclear cell layer in each such repetition to obtain a composite of mononuclear cell layer; and (d) culturing the composite of mononuclear cell layer obtained in step (c) in StemPro MSC SFM XenoFree medium with 1% volume by volume (V/V) of non-essential amino acid, ng/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

An alternate embodiment of the in vitro produced human pluripotent stem cells (HPSCs) for insulin production as disclosed herein, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acid, 1:1000 volume by volume (V/V) ratio of 2-Mercaptoethanol to induction medium, long/ml basal fibroblast growth factor (bFGF), 3 ng/ml weight by volume (W/V) of interleukin-3, and 5 ng/ml weight by volume (W/V) of interleukin-17.

An alternate embodiment of the in vitro produced human pluripotent stem cells (HPSCs) for insulin production as disclosed herein, wherein the induction medium for culturing the hADSCs is changed every other day till the hADSCs reach confluence before passages up to the at least five passages referred to as P5 to obtain the P5 hADSCs, and wherein the hADSCs until obtaining of said P5 hADSCs are grown in 10-cm cell culture dishes coated with 0.1% weight by volume (W/V) of gelatin.

An alternate embodiment of the in vitro produced human pluripotent stem cells (HPSCs) for insulin production as disclosed herein, wherein the TRA-1-60 positive cells referred to as dgHPSCs can be viably passaged for at least twenty passages.

An embodiment of the present disclosure provides an in vitro method for producing human pluripotent stem cells (HPSCs) for insulin production in a glucose-concentration responsive manner, the method comprising the steps of: (i) obtaining a lipoaspirate from a human; (ii) preparing human adipose-derived stem cells (hADSCs) from the lipoaspirate of step (i) involving isolation and single cell clone selection of adipose stem cells to obtain a selected clone followed by proliferation and expansion of cells of the selected clone to obtain the hADSCs; (iii) inducing and growing the hADSCs from step (ii) in an induction medium for at least five passages referred to as P5 to obtain P5 hADSCs; (iv) culturing the P5 hADSCs of step (iii) in the induction medium without applying any genetic engineering techniques to obtain cultured cells and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs of step (iii) referred to as directly-generated human pluripotent stem cells (dgHPSCs); and (v) genetically engineering the dgHPSCs of step (iv) by transduction of said dgHPSCs with the human estrogen-related receptor gamma (ERRγ) gene by a lentivirus vector, pWPI/EERγ encoding the human ERRγ gene to obtain transduced dgHPSCs referred to as dgHPSCs+ERRγ cells, which are the HPSCs for insulin production.

An alternate embodiment of the in vitro method for producing human pluripotent stem cells (HPSCs) for insulin production in a glucose-concentration responsive manner as disclosed herein, wherein the lipoaspirate from a human of step (i) as disclosed hereinabove is obtained by collecting a lipoaspirate by an abdominal adipose tissue liposuction procedure performed in a volunteer human donor.

An alternate embodiment of the in vitro method for producing human pluripotent stem cells (HPSCs) for insulin production in a glucose-concentration responsive manner as disclosed herein, wherein the human adipose-derived stem cells (hADSCs) are prepared in step (ii) as disclosed hereinabove from the lipoaspirate of step (i) as disclosed hereinabove by a method comprising the steps of: (a) washing the lipoaspirate of step (i) as disclosed hereinabove with Dulbecco's phosphate buffered saline (DPBS) and centrifuging them to remove red blood cells into the suspension and for obtaining a cell pellet; (b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube; (c) repeating the digestion with collagenase as in step (b) until the lipoaspirate of step (i) as disclosed hereinabove is completely digested and to isolate mononuclear cell layer in each such repetition to obtain a composite of mononuclear cell layer; and (d) culturing the composite of mononuclear cell layer obtained in step (c) in StemPro MSC SFM XenoFree medium with 1% volume by volume (V/V) of non-essential amino acid, ng/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

An alternate embodiment of the in vitro method for producing human pluripotent stem cells (HPSCs) for insulin production in a glucose-concentration responsive manner as disclosed herein, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acids, 1:1000 volume by volume (V/V) ratio of 2-Mercaptoethanol to induction medium, 10 ng/ml basal fibroblast growth factor (bFGF), 3 ng/ml weight by volume (W/V) of interleukin-3, and 5 ng/ml weight by volume (W/V) of interleukin-17.

An alternate embodiment of the in vitro method for producing human pluripotent stem cells (HPSCs) for insulin production in a glucose-concentration responsive manner as disclosed herein, wherein the induction medium in step (iii) as disclosed hereinabove is changed every other day till the hADSCs reach confluence before passages up to the at least five passages referred to as P5 to obtain the P5 hADSCs, and wherein the hADSCs of step (iii) as disclosed hereinabove until obtaining of said P5 hADSCs are grown in 10-cm cell culture dishes coated with 0.1% weight by volume (W/V) of gelatin.

An alternate embodiment of the in vitro method for producing human pluripotent stem cells (HPSCs) for insulin production in a glucose-concentration responsive manner as disclosed herein, wherein dgHPSCs+ERRγ cells are cultured in the Dulbecco's Modified Eagle Medium (DMEM) and 10% volume by volume (V/V) of fetal bovine serum (FBS) and treated with glucose at 5.5 mmol/L and 25 mmol/L of glucose at 37° C. for 4 hours to quantify the level of human insulin secretion for assessing the insulin production in a glucose-concentration responsive manner.

The invention will be further explained by the following Examples, which are intended to purely exemplary of the invention, and should not be considered as limiting the invention in any way.

### EXAMPLES

### Example 1—Production of Human Pluripotent Stem Cells (HPSCs) from Human Adipose-Derived Stem Cells (hADSCs)

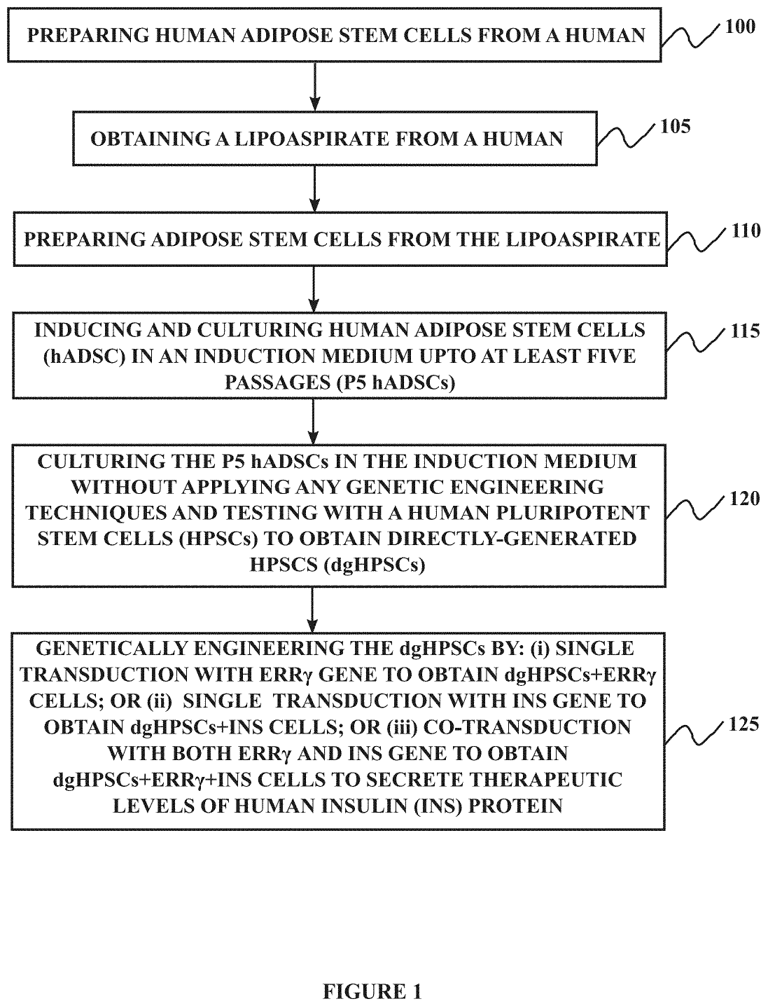

In this example, as illustrated partially in the flow-chart of FIG. 1 the production of human pluripotent stem cells (HPSCs) from human adipose stem cells (hADSCs) without any genetic engineering techniques in the presence of a specific induction medium to obtain directly-generated human pluripotent stem cells (dgHPSCs) is disclosed.

FIG. 1 illustrates the process steps for the method for engineering human pluripotent stem cells for insulin production and potential diabetes therapy in the future with such stem cells by co-transduction as per the present disclosure. At Step 100, hADSCs are prepared from a human donor. As shown in FIG. 1, to achieve Step 100, lipoaspirate is obtained from a human donor (Step 105) and human adipose-derived stem cells are prepared from the lipoaspirate (Step 110). In the present disclosure, hADSCs were prepared via single-cell clone selection of the cells possessing hADSC markers, followed by expansion of the selected cells to ensure non-hADSC cells can be removed. Thus, in the present disclosure, the lipoaspirate was collected by standard abdominal adipose tissue liposuction procedure from human volunteer donors. The lipoaspirate tissue was washed using Dulbecco's phosphate buffered saline (DPBS) and centrifuged to remove red blood cells. The resulting pellet was digested at 37° C. for 30 mins with 0.1% collagenase, followed by centrifugation at 800 g for 20 mins to isolate mononuclear cell layer which was a white membrane layer on the top of the liquid in the centrifuge tube.

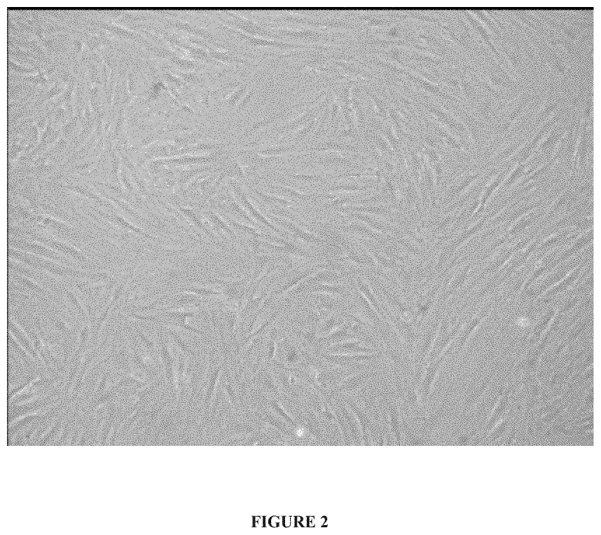

The aforementioned collagenase digestion was repeated until lipoaspirate tissue was completely digested. The isolated mononuclear cells were cultured under StemPro MSC SFM XenoFree medium with 1% non-essential amino acid, 10 ng/mL SCF, and 1% ITS (GIBCO, USA) for 48 hours, and the cells were passaged by limiting dilution. The diluted cell suspension was transferred into 96-well plates for cell expansion. Single-cell clones were selected by microscopy, and the expended single-cell clones were further examined to verify the properties of adipose-derived stem cells. FIG. 2 as disclosed herein depicts an inverted phase contrast microscope image of cell morphology of isolated mononuclear cells. Specifically, FIG. 2 depicts cell morphology using an inverted phase contrast microscope. The image shows that the cultured cells were fusiform fibroblast-like and locally swirled.

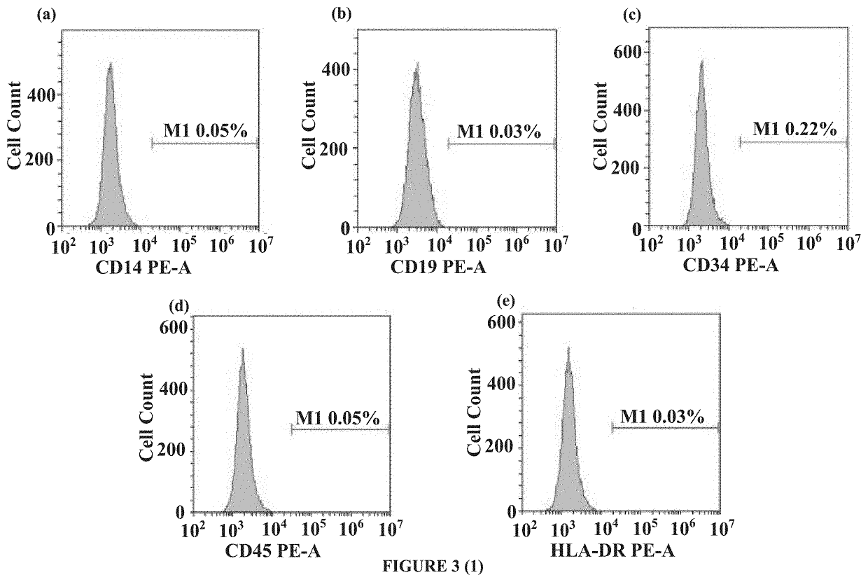

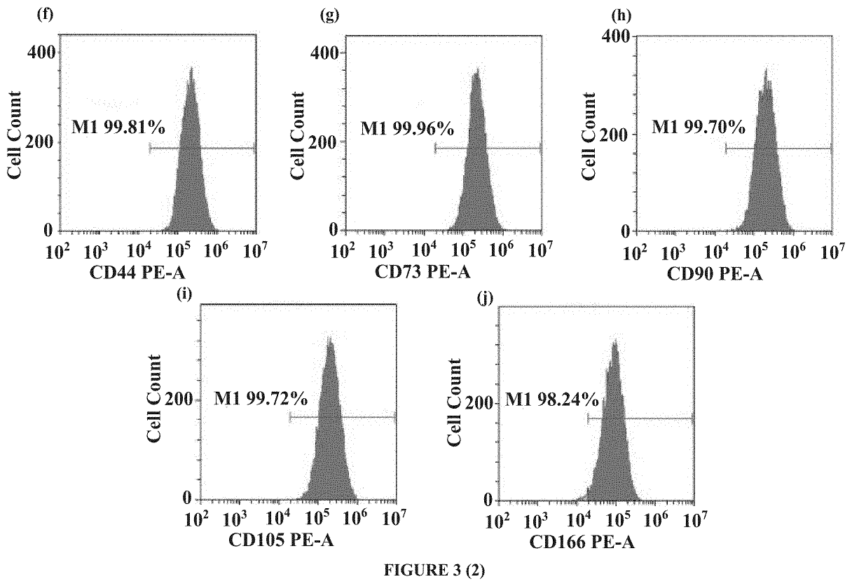

Next, to further identify if the cells derived and selected from adipose tissue possess hADSC phenotype, the fusiform fibroblast-like and locally swirled cells were analyzed by flow cytometry using a series of antibodies against the most commonly reported hADSCs cell surface markers (refer, Mildmay-White et al.). The cells were labeled with R-phycoerythrin (PE)-conjugated antibodies against human CD14, CD19, CD34, CD45, HLA-DR, CD44, CD73, CD90, CD105, and CD166 (Becton-Dickinson Biosciences, San Jose, Calif., USA) for 30 min on ice, washed with PBS containing 1% (w/v) BSA and total 10,000 cells/each labeling were analyzed by flow cytometry using FACS Caliber (Becton-Dickinson Biosciences, San Jose, Calif., USA). FIG. 3A and FIG. 3B illustrate the results of this phenotypic characterization where the hADSCs were CD14 (panel (a)), CD19 (panel (b)), CD34 (panel (c)), CD45 (panel (d)), and HLA-DR (panel (e)) negative in FIG. 3A; and CD44 (panel (f)), CD73 (panel (g)), CD90 (panel (h)), CD105 (panel (i)), and CD166 (panel (j)) positive in FIG. 3B. “M1” identified in the flow cytometry histogram in FIG. 3A and FIG. 3B is a marker placed to designate the range for positive peaks.

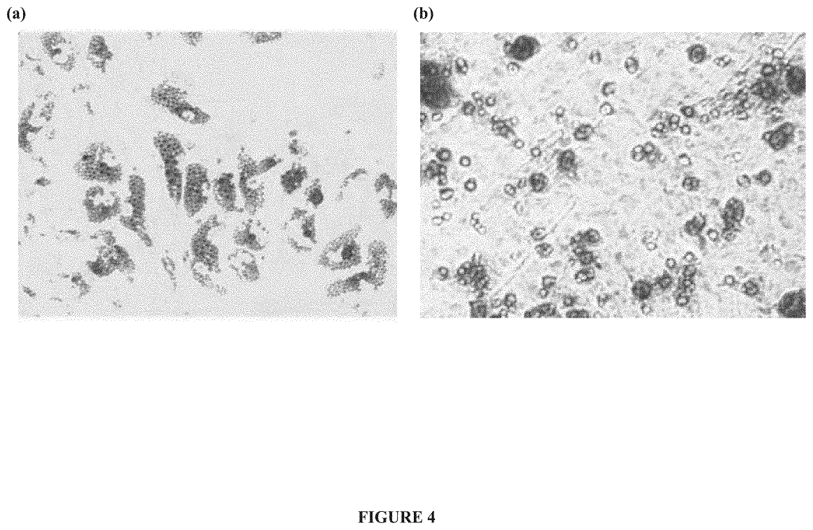

Additional experiments were undertaken to verify the isolation and proliferation of adipose-derived stem cells. First, adipocyte differentiation potential was verified. Here, the passage 3 (P3) of morphologically and phenotypically identified cells as described above were transferred to an adipogenic induction medium containing base medium DMEM, 10% fetal bovine serum, 0.5 mM IBMX (isobulyl-1-methylxanthione), 0.1 mM indomethacin and 111M dexamethasone (Sigma USA). Oil red 0 staining was performed on day 6, 12, and 16. The results showed vacuoles having an increased refractive index filled in the whole cell at day 12 after incubation with the aforementioned induction medium. Oil red 0 staining revealed 60-80% of the cells in the culture were rich in fat droplets (refer, panel (a) of FIG. 4) suggesting that the cells possessed adipocyte differentiation potential in vitro.

Next, osteoblasts differentiation potential in vitro was verified. Here, the passage 3 (P3) of the morphologically and phenotypically identified cells were cultured in 6-well plates with osteogenic induction medium which contained basic medium DMEM, 10% fetal bovine serum, 10 mm-glycerin phosphate, 0.1 mM ascorbic acid phosphate Vc, and 0.1 μm dexamethasone. The medium was changed every three days and the cells were cultured for up to 3 weeks. Alizarin red staining was performed at week 2 and week 3 respectively. The results showed that the cells in the osteogenic induction culture were transformed from the spindle-like shape to cubic-like and formed a multi-layer nodule structure. Two weeks after induction, mineralized nodules were formed; and after 3 weeks, more nodules and intense Alizarin red stain could be observed (refer, panel (b) of FIG. 4). These results suggest that the adipose-derived stem cells of the present disclosure possessed osteoblasts differentiation potential in vitro.

Further, as provided in FIG. 1 at Step 115, the hADSCs were induced and cultured in the presence of a specific induction medium up to five passages (P5) to obtain P5 hADSCs. Further, as provided in FIG. 1 at Step 120, the P5 hADSCs were cultured in the induction medium as disclosed herein without applying any genetic engineering techniques and then tested with a human pluripotent stem cell (HPSC) marker, TRA-1-60 immuno-fluorescent stain. The induction medium for the present disclosure was prepared using Knockout DMEM (Gibco, USA) as the base medium with the additions of 20% Knockout serum replacement (KSR) (Gibco, USA), 280 μg/ml L-glutamine (Gibco, USA), 5 ng/ml Arginine (Sigma, USA), 1% 100× MEM-nonessential amino acid (Gibco, USA), 1:1000 2-Mercaptoethanol (Sigma USA), ng/ml bFGF (basal fibroblast growth factor, PeproTech, USA), 3 ng/ml interleukin-3 (Sigma, USA), and 5 ng/ml interleukin-17 (Sigma, USA).

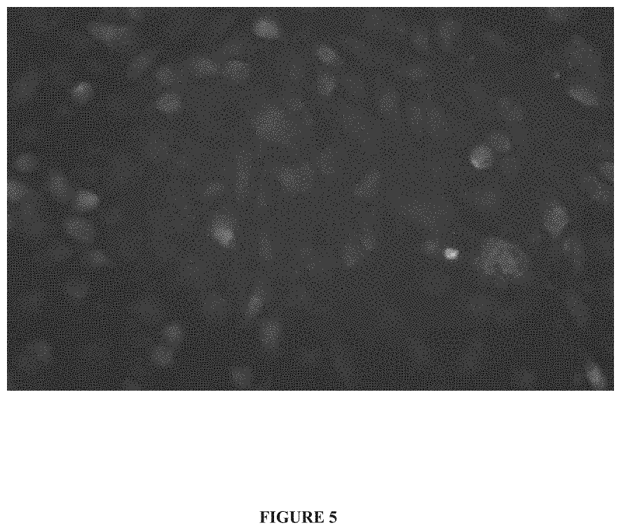

ADSCs were seeded and cultured with the aforementioned induction medium as described above in 10-cm cell culture dishes coated with 0.1% (w/v) gelatin (Gibco, USA). The induction medium was changed every other day until the cells completely reached confluence. The confluent cells were then passaged at a splitting ratio of 1:5 and continually cultured and inducted with medium change every other day. At passage 5, i.e., P5 ADSCs (which was for a total of about 20 days), the cells were tested for reprogrammed pluripotent stem cell colonies by TRA-1-60 immuno-fluorescent stain, for instance, as illustrated by a representative example in FIG. 5. TRA-1-60 live staining was used for identifying successfully reprogrammed colonies, based on a previous report that along with assessing morphological differences, staining with TRA-1-60-specific antibody can be used to distinguish successfully reprogrammed colonies from other transformed non-iPSC colonies. Briefly, in the present disclosure, the induced cells were cultured in a 0.1% (w/v) gelatin-coated 24-well plate, and were incubated with 1:300 mouse IgM anti-TRA-1-60 (Millipore, USA) followed by washing and 1:500 Alexa Fluor 555-conjugated secondary goat anti-mouse IgM antibody (Invitrogen, USA) in the induction medium at 37° C., 5% CO₂ for 1 hour, washed twice with PBS and replaced with 1 ml induction medium and examined under a fluorescent microscope. The TRA-1-60 positive cells were therefore determined to be dgHPSCs of the present invention. It has been confirmed in the present disclosure that the dgHPSCs of the present disclosure can be passaged for up to more than 20 passages.

### Example 2—Production of Insulin from dgHPSCs Singly Transduced or Co-Transduced with the Human Estrogen-Related Receptor Gamma (ERRγ) Gene or/and with the Human Insulin (INS) Gene

In this example, as illustrated partially in the flow-chart of FIG. 1, in step 125 the above-obtained directly-generated human pluripotent stem cells (dgHPSCs) of Example 1 were genetically engineered by single- or co-transduction of the aforesaid dgHPSCs with the human estrogen-related receptor gamma (ERRγ) gene (Genbank ID: NM_001134285.2) or/and with the human insulin (INS) gene (Genbank ID: NM_000207.3) for insulin production and secretion of potentially therapeutic levels of insulin protein as disclosed herein.

For the present disclosure, the insulin lentivirus gene vector, pWPI/INS and the ERRγ lentivirus gene vector, pWPI/ERRγ were constructed using original vector pWPI/hPLKWT/Neo (Addgene plasmid #35385). Briefly, the DNAs of pWPI/hPLKWT/Neo vector and insert genes, human insulin and ERRγ, were digested with BamHI restriction enzyme (New England Biolabs, USA), separated by 1% Agarose Gel Electrophoresis, and recovered by QIAGEN Gel Extraction kit (QIAGEN, Germany). The recovered pWPI vector and inserts were ligated with T4 DNA ligase (New England BioLabs) respectively and transformed into Top 10 competent cells according to the manufacturer's instructions (Invitrogen, USA). The positive colonies were primarily analyzed by BamHI digestion and further confirmed by DNA sequencing.

The pWPI/INS lentiviruses or pWPI/ERRγ lentiviruses were produced in HEK293T cells by transfection of pWPI/INS or pWPI/ERRγ lentivirus vectors, respectively, with packaging vectors psPAX2 (Addgene plasmid #12260) and pMD2.G (Addgene plasmid #12259) using Lipofectamine 2000. Briefly, HEK293T cells were cultured until 90% confluence, the transfer vectors, pWPI/INS or pWPI/ERRγ with the packaging vectors, psPAX2 and pMD2.G, respectively, were co-transfected by Lipofectamine 2000 according to the manufacturer's instructions. The cells were incubated at 37° C., 5% CO₂ incubator for at least 6 hours or overnight. Transfection reagents were removed and the cells were incubated with normal culture medium for 48 hours after transfection. The supernatant was collected and centrifuged at 2000 g for 20 minutes and then filtrated via 0.45 μm filters.

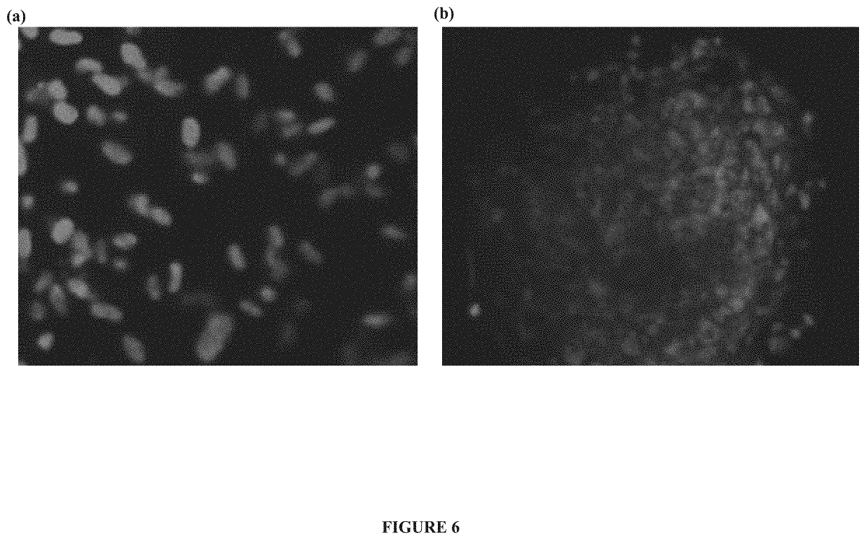

The dgHPSCs were cultured in 15-cm dishes with DMEM (Dulbecco's Modified Eagle Medium) and 10% fetal bovine serum. After removal of cell culture medium, pWPI/INS lentiviruses or pWPI/ERRγ lentiviruses or a mix of both pWPI/INS and pWPI/ERRγ lentiviruses in 25 ml filtrated cell culture medium of the transfected HEK293T cells were added into each dish and incubated for 6 hours to overnight to transduce the dgHPSCs. Insulin secretion of dgHPSCs was detected 24 hours after infection with pWPI/INS or pWPI/ERRγ lentiviruses or co-infection with mix of pWPI/INS and pWPI/ERRγ lentiviruses. Insulin secretion peaked 72 h after infection as revealed by insulin fluorescent immunochemistry stain as shown in FIG. 6. As shown therein, panel (a) of FIG. 6 demonstrates the DAPI nuclear stain in the nucleus of dgHPSC cells but without any immunofluorescent TRITC stain for insulin in the cytoplasm of the non-transduced dgHPSC cells indicating no insulin production in such cells. Whereas, panel (b) of FIG. 6 demonstrates both the staining with the DAPI nuclear stain in the nucleus of pWPI/ERRγ transduced dgHPSC cells along with positive immunofluorescence with TRITC stain for insulin in the cytoplasm of these transduced dgHPSC cells indicating insulin production in the transduced cells.

In the present disclosure, the dgHPSCs cells were infected with pWPI/ERRγ lentiviruses or pWPI/INS lentiviruses, or both pWPI/ERRγ and pWPI/INS lentiviruses. Two days post infection, insulin in the cell culture supernatants was tested. The concentration of insulin in the supernatant of pWPI/ERRγ lentiviruses infected dgHPSCs (dgHPSCs+ERRγ) was 30.84 μIU/ml, whereas that of pWPI/INS lentiviruses infected dgHPSCs (dgHPSCs+INS) was 11.61 μIU/ml and that of pWPI/ERRγ plus pWPI/INS lentiviruses infected dgHPSCs (dgHPSCs+ERRγ+INS) was 84.47 μIU/ml which was much higher than that of the dgHPSCs+INS cells or dgHPSCs+ERRγ cells.

### Example 3—Production of Insulin in a Glucose-Concentration Responsive Manner from dgHPSCs Singly Transduced with the Human Estrogen-Related Receptor Gamma (ERRγ) Gene

In this example, forced expression of ERRγ gene by lentivirus vector pWPI/ERRγ transduction in dgHPSC cells demonstrated insulin production in a glucose-responsive manner. pWPI/EERγ transduced dgHPSC cells were cultured in the DMEM (Dulbecco's Modified Eagle Medium) and 10% fetal bovine serum with 5.5 and 25 mmol/L glucose respectively at 37° C. for 4 hours. The quantitation of insulin secretion in the supernatant was analyzed by an insulin enzyme-linked immunoassay kit (DiaMetra, Cat #DCM076-8, Italy) following the assay instruction of the manufacturer. The insulin concentration in the supernatant of the cells cultured under 5.5 mmol/L glucose was 3.46 μU/ml, whereas that of the cells cultured under 25 mmol/L glucose increased to 11.95 μU/ml. This result supports the role of ERRγ in regulating insulin secretion in a glucose-responsive manner and suggests that engineering the gene into stem cells may be a promising approach for diabetes therapy.

Therefore, human pluripotent stem cells overexpressing ERRγ can efficiently synthesize and secrete human insulin at the pluripotent stem cell state, do not need to differentiate into β-like cells; and in addition, co-expression of human insulin and ERRγ genes can synergistically further promote synthesis and secretion of human insulin in dgHPSC cells. Insulin quantities secreted into cell culture supernatant was tested by an electrochemiluminescence method performed by Kingmed Diagnostics (Jinan, China). Human pluripotent stem cells overexpressing ERRγ or co-expression of human insulin and ERRγ preferably can potentially be introduced into humans for potential diabetes therapy in the future.

Although the disclosure has been explained in relation to its preferred embodiment, it is to be understood that many other possible modifications and variations can be made without departing from the spirit and scope of the disclosure. It is intended that the specification and examples be considered as exemplary only, with a true scope and spirit of the invention being indicated by the following claims.

### REFERENCES

1. Sun N. et al., Feeder-free derivation of induced pluripotent stem cells from adult human adipose stem cells. Proc Natl Acad Sci USA. 106(37):15720-1525, 2009.
2. Qu X. et al., Induced Pluripotent Stem Cells Generated from Human Adipose-Derived Stem Cells Using a Non-Viral Polycistronic Plasmid in Feeder-Free Conditions. Plos One, 7(10): e48161, 2012.
3. Wang Z. et al., Detection of integration of plasmid DNA into host genomic DNA following intramuscular injection and electroporation. Gene Therapy, 11: 711-721, 2004.
4. Mildmay-White A. et al., Cell Surface Markers on Adipose-Derived Stem Cells: A Systematic Review. Curr Stem Cell Res Ther. 12(6):484-492, 2017.
5. Klimczak M, Oncogenesis and induced pluripotency—commonalities of signaling pathways, Contemp Oncol (Pozn). 19(1A): A16-A21, 2015.

What is claimed:

1. An in vitro method for producing human pluripotent stem cells (HPSCs) for insulin production, the method comprising the steps of:
(i) obtaining a lipoaspirate from a human;
(ii) preparing human adipose-derived stem cells (hADSCs) from the lipoaspirate of step (i) involving isolation and single cell clone selection of adipose stem cells to obtain a selected clone followed by proliferation and expansion of cells of the selected clone to obtain the hADSCs;
(iii) inducing and growing the hADSCs from step (ii) in an induction medium for at least five passages referred to as P5 to obtain P5 hADSCs;
(iv) culturing the P5 hADSCs of step (iii) in the induction medium without applying any genetic engineering techniques to obtain cultured cells and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs of step (iii) referred to as directly-generated human pluripotent stem cells (dgHPSCs);
(v) genetically engineering the dgHPSCs of step (iv) in a manner selected from a group consisting of: by transduction of said dgHPSCs with the human estrogen-related receptor gamma (ERRγ) gene by a lentivirus vector, pWPI/ERRγ encoding the human ERRγ gene to obtain transduced dgHPSCs referred to as dgHPSCs+ERRγ cells, which are first of the groups of HPSCs for insulin production, and by co-transduction of said dgHPSCs with said ERRγ gene by the lentivirus vector pWPI/ERRγ and with the said INS gene by a lentivirus vector, pWPI/INS encoding the human INS gene to simultaneously introduce the human ERRγ gene and the human INS gene in the dgHPSCs of step (iv) to obtain co-transduced dgHPSCs referred to as dgHPSCs+ERRγ+INS cells, which are second of the groups of HPSCs for insulin production,
wherein the co-transduced dgHPSCs referred to as dgHPSCs+ERRγ+INS cells show co-expression of the human ERRγ gene and the human INS gene to synergistically promote the synthesis and secretion of human insulin at the stem cell state, and
wherein the co-transduced dgHPSCs referred to as dgHPSCs+ERRγ+INS cells transduced with both the human ERRγ gene and the human INS gene secrete a higher level of human insulin compared to dgHPSCs transduced with only the human ERRγ gene referred to as dgHPSCs+ERRγ cells.

2. The in vitro method of claim 1, wherein the lipoaspirate from a human of step (i) of claim 1 is obtained by collecting a lipoaspirate by an abdominal adipose tissue liposuction procedure performed in a volunteer human donor.

3. The in vitro method of claim 1, wherein the human adipose-derived stem cells (hADSCs) are prepared in step (ii) of claim 1 from the lipoaspirate of step (i) of claim 1 by a method comprising the steps of:
(a) washing the lipoaspirate of step (i) of claim 1 with Dulbecco's phosphate buffered saline (DPBS) and centrifuging them to remove red blood cells into the suspension and for obtaining a cell pellet;
(b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube;
(c) repeating the digestion with collagenase as in step (b) until the lipoaspirate of step (i) is completely digested and to isolate mononuclear cell layer in each such repetition to obtain a composite of mononuclear cell layer; and
(d) culturing the composite of mononuclear cell layer obtained in step (c) in StemPro MSC SFM XenoFree medium with 1% volume by volume (V/V) of non-essential amino acid, ng/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

4. The in vitro method of claim 1, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acids, 1:1000 volume by volume (V/V) ratio of 2-Mercaptoethanol to induction medium, 10 ng/ml basal fibroblast growth factor (bFGF), 3 ng/ml weight by volume (W/V) of interleukin-3, and 5 ng/ml weight by volume (W/V) of interleukin-17.

5. The in vitro method of claim 1, wherein the induction medium in step (iii) of claim 1 is changed every other day till the hADSCs reach confluence before passages up to at least five passages referred to as P5 to obtain the P5 hADSCs, and wherein the hADSCs of step (iii) of claim 1 until obtaining of said P5 hADSCs are grown in 10-cm cell culture dishes coated with 0.1% weight by volume (W/V) of gelatin.

6. The in vitro method of claim 1, wherein the dgHPSCs of step (iv) of claim 1 can be viably passaged for at least twenty passages.

7. An in vitro produced human pluripotent stem cells (HPSCs) for insulin production comprising: HPSCs produced from human adipose-derived stem cells (hADSCs) by treating said hADSCs with an induction medium without applying any genetic engineering techniques for at least five passages referred to as P5 to obtain P5 hADSCs and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs referred to as directly-generated human pluripotent stem cells (dgHPSCs); and
co-transduction of said dgHPSCs with the human estrogen-related receptor gamma (ERRγ) gene by the lentivirus vector pWPI/ERRγ encoding the human ERRγ gene and with human insulin (INS) gene by a lentivirus vector, pWPI/INS encoding the human INS gene to simultaneously introduce the human ERRγ gene and the human INS gene in the dgHPSCs to obtain co-transduced dgHPSCs referred to as dgHPSCs+ERRγ+INS cells,
wherein the hADSCs are produced from a lipoaspirate from a human involving isolation and single cell clone selection of adipose stem cells to obtain a selected clone followed by proliferation and expansion of cells of the selected clone to obtain the hADSCs,
wherein the dgHPSCs are alternatively genetically engineered by transduction of said dgHPSCs with the human estrogen-related receptor gamma (ERRγ) gene only by a lentivirus vector, pWPI/EERγ encoding the human ERRγ gene to obtain transduced dgHPSCs referred to as dgHPSCs+ERRγ cells, and
wherein the co-transduced dgHPSCs referred to as dgHPSCs+ERRγ+INS cells transduced with both the human ERRγ gene and the human INS gene secrete a higher level of human insulin compared to dgHPSCs transduced with only the human ERRγ gene referred to as dgHPSCs+ERRγ cells.

8. The dgHPSCs of claim 7, wherein the lipoaspirate from a human is obtained by collecting a lipoaspirate by an abdominal adipose tissue liposuction procedure performed in a volunteer human donor.

9. The dgHPSCs of claim 7, wherein the human adipose-derived stem cells (hADSCs) are prepared from the lipoaspirate by a method comprising the steps of:
(a) washing the lipoaspirate with Dulbecco's phosphate buffered saline (DPBS) and centrifuging them to remove red blood cells into the suspension and for obtaining a cell pellet;
(b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube;
(c) repeating the digestion with collagenase as in step (b) until the whole of the lipoaspirate is completely digested and to isolate mononuclear cell layer in each such repetition to obtain a composite of mononuclear cell layer; and
(d) culturing the composite of mononuclear cell layer obtained in step (c) in StemPro MSC SFM XenoFree medium with 1% volume by volume (V/V) of non-essential amino acid, ng/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

10. The dgHPSCs of claim 7, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acid, 1:1000 volume by volume (V/V) ratio of 2-Mercaptoethanol to induction medium, 10 ng/ml basal fibroblast growth factor (bFGF), 3 ng/ml weight by volume (W/V) of interleukin-3, and 5 ng/ml weight by volume (W/V) of interleukin-17.

11. The dgHPSCs of claim 7, wherein the induction medium for culturing the hADSCs is changed every other day till the hADSCs reach confluence before passages up to the at least five passages referred to as P5 to obtain the P5 hADSCs, and wherein the hADSCs until obtaining of said P5 hADSCs are grown in 10-cm cell culture dishes coated with 0.1% weight by volume (W/V) of gelatin.

12. The dgHPSCs of claim 7, wherein the TRA-1-60 positive cells referred to as dgHPSCs can be viably passaged for at least twenty passages.

13. An in vitro method for producing human pluripotent stem cells (HPSCs) for insulin production in a glucose-concentration responsive manner, the method comprising the steps of:
(i) obtaining a lipoaspirate from a human;
(ii) preparing human adipose-derived stem cells (hADSCs) from the lipoaspirate of step (i) involving isolation and single cell clone selection of adipose stem cells to obtain a selected clone followed by proliferation and expansion of cells of the selected clone to obtain the hADSCs;
(iii) inducing and growing the hADSCs from step (ii) in an induction medium for at least five passages referred to as P5 to obtain P5 hADSCs;
(iv) culturing the P5 hADSCs of step (iii) in the induction medium without applying any genetic engineering techniques to obtain cultured cells and testing the cultured cells with a human pluripotent stem cells (HPSCs) markers including TRA-1-60 immuno-fluorescent stain to obtain TRA-1-60 positive cells, wherein said TRA-1-60 positive cells are HPSCs derived from P5 hADSCs of step (iii) referred to as directly-generated human pluripotent stem cells (dgHPSCs); and
(v) genetically engineering the dgHPSCs of step (iv) by transduction of said dgHPSCs with the human estrogen-related receptor gamma (ERRγ) gene by a lentivirus vector, pWPI/EERγ encoding the human ERRγ gene to obtain transduced dgHPSCs referred to as dgHPSCs+ERRγ cells, which are the HPSCs for insulin production.

14. The in vitro method of claim 13, wherein the lipoaspirate from a human of step (i) of claim 13 is obtained by collecting a lipoaspirate by an abdominal adipose tissue liposuction procedure performed in a volunteer human donor.

15. The in vitro method of claim 13, wherein the human adipose-derived stem cells (hADSCs) are prepared in step (ii) of claim 13 from the lipoaspirate of step (i) of claim 13 by a method comprising the steps of:
(a) washing the lipoaspirate of step (i) of claim 13 with Dulbecco's phosphate buffered saline (DPBS) and centrifuging them to remove red blood cells into the suspension and for obtaining a cell pellet;
(b) digesting the cell pellet of step (a) at 37° C. for 30 mins with 0.1% weight by volume (W/V) of collagenase and centrifuging them at 800 g for 20 mins to isolate mononuclear cell layer which is a white membrane layer on top of the liquid in a centrifuge tube;
(c) repeating the digestion with collagenase as in step (b) until the lipoaspirate of step (i) of claim 13 is completely digested and to isolate mononuclear cell layer in each such repetition to obtain a composite of mononuclear cell layer; and
(d) culturing the composite of mononuclear cell layer obtained in step (c) in StemPro MSC SFM XenoFree medium with 1% volume by volume (V/V) of non-essential amino acid, long/mL weight by volume (W/V) of human stem cell factor (SCF) and 1% volume by volume (V/V) of Insulin, Transferrin, Selenium (ITS) cell culture supplement for a time period in a range of 2 weeks to 3 weeks by passaging for 2 to 3 passages to obtain the human adipose-derived stem cells (hADSCs).

16. The in vitro method of claim 13, wherein the induction medium comprises Knockout Dulbecco's Modified Eagle Medium (DMEM) as a base medium with the additions of 20% volume by volume (V/V) of Knockout serum replacement (KSR), 280 μg/ml weight by volume (W/V) of L-glutamine, 5 ng/ml weight by volume (W/V) of Arginine, 1% volume by volume (V/V) of 100× Minimum Essential Medium (MEM)-nonessential amino acids, 1:1000 volume by volume (V/V) ratio of 2-Mercaptoethanol to induction medium, long/ml basal fibroblast growth factor (bFGF), 3 ng/ml weight by volume (W/V) of interleukin-3, and 5 ng/ml weight by volume (W/V) of interleukin-17.

17. The in vitro method of claim 13, wherein the induction medium in step (iii) of claim 13 is changed every other day till the hADSCs reach confluence before passages up to the at least five passages referred to as P5 to obtain the P5 hADSCs, and wherein the hADSCs of step (iii) of claim 13 until obtaining of said P5 hADSCs are grown in 10-cm cell culture dishes coated with 0.1% weight by volume (W/V) of gelatin.

18. The in vitro method of claim 13, wherein dgHPSCs+ERRγ cells are cultured in the Dulbecco's Modified Eagle Medium (DMEM) and 10% volume by volume (V/V) of fetal bovine serum (FBS) and treated with glucose at 5.5 mmol/L and 25 mmol/L of glucose at 37° C. for 4 hours to quantify the level of human insulin secretion for assessing the insulin production in a glucose-concentration responsive manner.
